(19) United States
(12) Patent Application Publication
(10) Pub. No.: US 2011/0263710 A1
(43) Pub. Date: Oct. 27, 2011

(54) Transepithelial methods of using gamma aminobutyric acid compositions for pain relief

(76) Inventors: Michael H. Moskowitz, Mill Valley, CA (US); Marla DePolo Golden, Jacksonville, FL (US); Dana Gordon, Pebble Beach, CA (US)

(21) Appl. No.: 13/067,827

(22) Filed: Jun. 29, 2011

Related U.S. Application Data
(63) Continuation-in-part of application No. 12/692,935, filed on Jan. 25, 2010, now abandoned.

(51) Int. Cl. A61K 31/197; A61P 25/00

(52) U.S. Cl. 514/561

(57) ABSTRACT

Disclosed are methods for alleviating, relieving, inhibiting, and eliminating acute or chronic pain in a patient suffering from such pain by contacting epithelial tissue of the patient with exogenous GABA (gamma-amino butyric acid).

The epithelial tissue includes skin, nasal mucosa or membranes and other epithelial tissue such as buccal, palatal, sublingual, rectal, vaginal, thecal and the like. The GABA is supplied in compositions such as water, saline, buffered saline, parenteral solutions, lotions, semi-solids such as, gels, creams, pastes, salves, ointments, solids such as impregnated patches, impregnated rub-on solid compositions from which layers of GABA may be rubbed onto the skin, sublingual pills, suppositories and the like.

### CROSS REFERENCE TO RELATED APPLICATIONS

This application is a continuation-in-part of U.S. Ser. No. 12/692,935 entitled Transdermal Application of Gamma Amino Butyric Acid For Pain Relief, filed Jan. 25, 2010.

### STATEMENT REGARDING FEDERALLY SPONSORED RESEARCH OR DEVELOPMENT

(Not Applicable)

### STATEMENT REGARDING FEDERALLY SPONSORED RESEARCH OR DEVELOPMENT REFERENCE TO A SEQUENCE LISTING, A TABLE, OR A COMPUTER PROGRAM LISTING APPENDIX SUBMITTED ON COMPACT DISC (SEE 37 CFR 1.52 (e)(5))

(Not Applicable)

### BACKGROUND OF THE INVENTION

A. Field of the Invention

This invention relates to the relief or inhibition of chronic or acute pain by administering to a subject suffering from pain, a therapeutically effective amount of exogenous gamma aminobutyric acid (GABA) by contacting appropriate epithelial tissue of the subject.

More particularly, it relates to the application of formulations of GABA to epithelial tissue, primarily skin, nasal mucosa or oral mucosa, of a patient suffering from pain, to introduce pain-relieving amounts of exogenous GABA into the patient's body.

More particularly still, it relates to the application of exogenous GABA to the local site of a pain source of a subject to inhibit or relieve local pain at the affected site.

It also relates to the application of exogenous GABA to the nasal or oral cavity or mucosa or other epithelial tissue, such as the rectal or vaginal epithelium, of a subject suffering pain to inhibit, relieve or eliminate the pain of the subject systemically.

B. Prior Art

GABA is a naturally occurring polypeptide neurotransmitter present in the human body and also produced by tissue in response to an injury or other insult. It is utilized in the central and peripheral nervous systems. It is the chief inhibitory neurotransmitter in the central nervous system and the peripheral nervous system of the human and is known to modulate pain in the biochemistry of the human. It functions in balance with glutamate to alter the occurrence of pain and inhibition of pain depending on the amount of GABA released in response to the pain. GABA that occurs naturally in the body is referred to herein as endogenous GABA. GABA that is used in the invention is variously referred to as exogenous GABA, or simply as GABA when the context is clear that exogenous GABA is intended or apparent.

GABA and glutamate are the two main neurotransmitters in the brain at the base of balancing brain activity. Together these molecules account for 90% of all of the neurotransmitters in the brain. Simply put, glutamate turns on nerves and GABA turns them off. Generally, all chronic disease activity, including pain, in the brain involves the excitatory glutamate overwhelming the inhibitory GABA. GABA acts like a brake on glutamate to prevent excitation (and therefore, pain) from running away with the brain and communicates with the body to release its own excitatory (inhibiting) chemicals.

In the brain pathways responsible for controlling pain, GABA is often outmatched and glutamate dominates. As interactions between other processes in the brain shift toward this state of alert, more of the main pain neurotransmitter is released, leading to a cascade of networks of pain-processing cells. As pain processing grows there is an increase in the firing of nerves in the brain's pain networks, adding to the release of more inflammatory chemicals and hence more pain.

The body would normally respond in a case such as that just described, by releasing endogenous GABA from the body tissue involved in the injury. The GABA would act as a brake on excessively firing peripheral nerves and also work directly on inflammatory immune system cells to shut off the inflammation. In chronic pain subjects, the amount of GABA in the peripheral nervous system is not enough to shut off these nerves and the input persists.

The pain-suffering patient becomes increasingly helpless and feels that the only option is to suppress the pain. This is usually done by taking undesirable CNS suppressing pain killers that are only partially successful, leaving the patient to suffer with both the pain and the adverse CNS effects.

Exogenous GABA has been widely used in various forms and for various reasons. The compound is widely available over-the-counter in numerous forms including food products, such as rice and chocolate; drinks such as teas, coffees and sodas; oral supplements such as pills, tablets, powders and various liquid formulations; and topical versions such as creams, lotions, gels and the like.

The prior art variously describes exogenous GABA as being useful in a wide range of conditions and maladies, among which are: treatment of depression, sleep pattern disorders, anxieties, neuroses, hypertension, loss of libido, digestive disease and the like.

Despite the attribution of many properties to GABA in the prior art, some of which may or may not be factual, the prior art reports of various formulations containing GABA, various indications and the various routes of administration to patients, as will be discussed further hereinbelow, have not included any reports of the administration of exogenous GABA as set forth in the invention, to subjects suffering from pain for the purpose of reducing, inhibiting or eliminating the pain in such subjects.

### BRIEF SUMMARY OF THE INVENTION

The treatment of chronic or acute pain takes many forms and can be a lifelong battle using many different types of modalities. Some of these treatment modalities include injections, oral medications, patches and creams.

Although the treatment for the control of pain, particularly chronic pain, is necessary for the comfort of the patient, it also may carry with it some undesirable side effects. One of the common side effects is the impaired judgment that is caused by the pain medicine because most, if not all pain medicine, affects the person's brain. This side effect may produce impaired judgment, delayed reaction time, cognitive deficits, dependency, hallucinations, grogginess, and addiction among others.

In order for endogenous GABA to function in the body and act to relieve pain, properly functioning receptors are needed. In the person who is suffering from chronic pain, the endogenous GABA is overwhelmed as a result of spontaneous, persistent and excessive firing of the injured peripheral nerves. If there is not enough GABA to cover the receptors, the nerves that are affected continue to send signals to the brain and the pain level of the person increases and persists. The present invention surprisingly presents methods which deliver exogenous GABA to turn on the GABA receptors in the formerly continuously firing nerves to yield the pain relief described herein.

This invention thus comprises a method for alleviating pain in a subject suffering from pain, and in many cases eliminates pain for long periods of time, which comprises administering to the said subject suffering from pain, a therapeutically effective amount of exogenous GABA, to epithelial tissue, primarily to skin or the nasal or oral mucosa or membranes, or rectal or vaginal epithelium, but also to various other epithelial tissue as well.

The preferred application site is the local site of a pain source of a subject when it is desired to inhibit or relieve the local pain at the affected site. The preferred application site when it is desired to inhibit or relieve the pain of a subject systemically, is the nasal membranes or oral mucosa of the subject. As used herein, oral mucosa is meant to include buccal tissue, palatal tissue and sublingual tissue. Orally ingested GABA formulations which do not provide a therapeutically effective amount of GABA in contact with the oral cavity epithelial tissue are not included.

The GABA may be supplied in any convenient formulation. The compositions described in this specification in terms of physical forms thereof may range from freely flowing liquids such as water and other liquids of similar viscosity such as saline. sterile solutions for bladder, enteral, parenteral, epidural or intrathecal delivery, and the like, to more viscous liquids such as flowable lotions, to semi-solids which themselves may or may not be somewhat flowable but are layerable as a film or thin layer, such as creams, gels, pastes, salves, ointments, impregnated adhesive patches and the like, some of which may be of amorphous character or have no particular shape except that of their container, to solid forms of absorbable compositions applicable as a film or layer having the consistency of, for example, lipstick, lipbalms, rub-on deodorants and the like, and to solids such as rectal and vaginal suppositories, topical impregnated adhesive patches containing absorbable GABA formulations, dissolvable lozenges, sublingual pills, and the like.

The methods of the invention are practiced by bringing the appropriate GABA-containing formulation into contact with the epithelial tissue of the selected route of administration for a period of time sufficient to allow the GABA to be absorbed by the tissue.

The invention is thus directed to the discovery of a new use, i.e., that of pain alleviation of the known material, GABA.

### DETAILED DESCRIPTION OF THE INVENTION

To our knowledge, no one has described the actual use of GABA as a compound which can very quickly inhibit and ameliorate and eliminate pain, often for long periods of time, from a variety of sources in an affected subject. One aspect of the invention described herein, namely the application of GABA to the nasal cavity, has been found to create, within seconds, a systemic bodily response to pain which patients describe as a major, substantial reduction in the level of the pain which caused them to seek help in the first place.

In a second aspect of the invention, similar pain reduction has been achieved herein through the use of a formulation comprising GABA which is easily topically applied to the skin at a local site of the pain source.

The invention thus encompasses in one embodiment, a method of using exogenous GABA to inhibit the pain of a subject affected with said pain which comprises topically applying a therapeutically effective amount of exogenous GABA to the local site of the pain source, and in another embodiment, applying a therapeutically effective amount of GABA to the nasal cavity, or other internal site of epithelial tissue, in the case where systemic relief from pain is sought, and allowing the GABA to be absorbed at the site. As a result of the methods of the inventions, pain relief has been obtained by subjects ranging from significant and substantial reduction in pain to complete relief of pain for prolonged periods of time.

The preferred route of administration for some sources of pain from which systemic relief is sought, in this invention, is the intranasal route. The preferred formulation in this instance is a nasal spray of a solution of GABA in an appropriate aqueous vehicle or nasal drops of such a solution. A nasal spray is, of course, a collection of small drops and therefore, the term “nasal spray” as used herein, includes nasal drops. The preferred route of administration for the relief of local pain from local source is a topical skin composition such as a viscous liquid such as a lotion, or semi-solid cream, paste, salve, gel or similar cohesive form, or a solid form, which can be conveniently rubbed into, and adhere to, the skin, or a solid impregnated adhesive topical patch which adheres to the local site and from which GABA may be absorbed into the skin.

This is not to say that a viscous material could not be applied to the nasal or oral cavity or other epithelial tissue, or that a liquid could not be applied to a local site of pain, however. In each case, they may well be employed. Preferred, however is an aqueous spray vehicle for nasal use, as that form is more quickly absorbed by the nasal mucosa and enters the bloodstream more rapidly than would occur from a more viscous topical material.

Accordingly, the preferred route for administration, especially when a systemic effect is sought, is through the nasal cavity and the preferred formulation is a liquid nasal spray or unsprayed drops. The topical application to skin, however, is preferred for episodes of local pain origin such as muscle cramps, pain due to injury or exercise where pain is associated with a local causative source.

The utility and success of the invention may be achieved through the use of an almost boundless array of formulation. Various formulations for nasal sprays and drops, oral cavity sprays and drops, lozenges, sublingual pills, and other formulations, vaginal or rectal suppositories and topically applied skin formulations or compositions as vehicles for the GABA, such as solids, semi-solids, and liquid vehicles, abound. In addition, standard preparations for sterile injectable products may be used. Any of these may be employed provided they are not inhospitable to, or incompatible with, the GABA component and are consistent with the route of administration selected by the user.

The formulation may also contain active ingredients other than GABA although this is not necessarily preferred. Nor are any additional active ingredients necessary in the ability of GABA to relieve pain according to the invention. We have discovered that exogenous GABA alone is quite efficacious in relieving pain. Of course, the normal and usual excipients, moisturizers, adjuvants, preservatives and the like used in the art, may be employed as desired. These materials are too numerous to mention exhaustively, but the selection of specific ingredients is well within the skill of the art. Presented in the examples below are representative ingredients which have been sued in preparing compositions used in the invention. Merely as illustrative, any of the following materials alone or in various combinations are used in the art and may be used in the present invention. purified water, phospholipids, isopropyl myristate, cetearyl, alcohol, stearyl alcohol, cetyl alcohol, caprylic/capric triglyceride, ceteareth-20, wheat germ oil, glycerin, dimethicone, magnesium aluminum silicate, xanthan gum, polyacrylamide, C13-C14 isoparaffin, laureth-7, disodium EDTA, BHT (butylated hydroxytoluene), phenoxyethanol, methylisothiazolinone, methylchloroisothiazolinone, soya lecithin, isopropyl palmitate, sodium hydroxide, EDTA (edetate disodium), steric acid, glyceryl monostearate, simethicone, urea, polyoxyl 40 stearate, methylparaben, propylparaben, imidurea and ethyl alcohol.

The invention in its simplest and most direct form is quite accessible and comprises a method of providing a therapeutically effective amount of GABA in the formulation of the user's choice. In nasal or oral sprays, GABA dissolved in saline or even simply water, with or without a preservative, will be sufficient. In some cases, an oil base might be utilized in which event emulsifiers may be used as appropriate. In the topical skin form, GABA in a simple, skin formulation such as a cream, ointment, salve, paste or gel or any other semi-solid or solid vehicle as aforesaid, will serve quite well. The term “skin compositions” or “skin formulations”, or like terms as used herein are meant to apply to any such vehicle, irrespective of their specific non-GABA ingredients, whether included in the above list, or not.

The results of the invention are quite surprising and unexpected in view of the collected wisdom concerning accepted drug selection in the art. In this regard, it is generally thought that when bioactive materials, medicaments, drugs, and the like are sought, one looks to compounds that cross the blood-brain barrier in order to be systemically effective. GABA is known not to cross the blood-brain barrier, however, and therefore would not be expected to elicit a systemic response. The fact that it does create such an effect is of surprise in this invention, and the fact that the results in many cases last for a prolonged period of time after dosing is indeed unexpected. The art would have been motivated to avoid a compound of this type for the alleviation, inhibition or elimination of pain as described herein.

In conventional pain management, much of the medicine is absorbed into the bloodstream soon after being taken through the usual oral ingestion route. Because the conventional pain medicine enters the bloodstream, however, it will usually cross the blood-brain barrier because the molecules are designed to do so. Consequently, a major side effect of most pain medicines that are taken orally is the effect on the cognitive abilities of an individual because it has crossed the blood-brain barrier and impacted the functioning of the brain.

Although pain relief might be achieved, the patient nevertheless may suffer undesirable side effects, such as decreased immediate recall, decreased short term memory and decreased word finding abilities, as well as increased fatigue, drowsiness, confusion and memory disturbance. This is because once these medications cross the blood brain barrier, they are distributed throughout the whole brain, not just to the targeted sites.

In the present invention, the GABA formulation has a focused local effect when applied topically to the skin and because of the large size of the GABA molecule, is limited in its ability to cross the blood-brain barrier. Therefore, a principal advantage of the invention for both the topical application and the nasal or oral cavity contact route, is that pain-relief is obtained without loss of cognitive ability. Moreover, because the absorption is through the skin or the nasal or oral mucosa, the effect is often immediate in many cases and very rapid in others.

Another drawback to pain medicine which enters the blood stream is the length of time required for the medication to have the desired effect. Typically in orally supplied medicines for example, the medicine must be absorbed via blood vessels from the gastrointestinal tract, and eventually enters the bloodstream from that site. From the bloodstream, the medication must then go through another filtering process to cross the blood-brain barrier. There is thus, a significant delay between the ingestion of the pain medicine and the desired effect.

While we do not intend to be bound by any particular theory of the pharmacological effect of the invention, it appears that the brain, in some way, monitors the level of GABA present in the circulating blood-stream. When that concentration is high enough from the exogenously supplied GABA, the brain appears to send a signal throughout the body to turn off the excitatory nerves responsible for pain occurring anywhere in the body. The result is a systemic reduction, inhibition or even elimination of pain anywhere in the body where the nerves are firing, without regard to the cause or source. As has been noted above, the results are often apparent for prolonged periods of time even after dosing has been discontinued.

The invention thus involves routes of administration that result in somewhat different effects on pain, either systemic, as described in the previous paragraph, or local as further described below. These in turn tend to rely on how rich in capillaries the locus of GABA application may be.

For example, the nasal cavity is rich in the presence of capillary blood supply. Exogenously supplied GABA to that site, especially in the preferred concentrations of the invention, will be rapidly absorbed and appear almost instantaneously, usually within seconds, in the bloodstream. The brain then senses the higher amounts of GABA present in the bloodstream and sends a signal throughout the system to turn off the excited nerves which caused the pain. As a result, the patient experiences pain relief. Similar blood supply exists in sublingual, buccal and palatal tissue contact and similar effects on pain are obtained when that form of the oral route of administration is used.

In local areas of topical skin administration to skin and other areas not as rich in capillary blood supply as the nasal cavity, the exogenously added GABA tends to concentrate in the local areas where it overwhelms the pain-inducing effect of inflammation or nerve injury on the excitatory nerves, reducing the electrical signals of the local nerves. Thus, local relief from the source of pain at the local site is obtained without ever increasing substantially the concentration of GABA in the bloodstream or the brain. This is so even though the amount of GABA introduced per dose topically exceeds the dose introduced intranasally, as will be described in more detail below.

In topical skin application of the present invention, instead of entering the bloodstream, the GABA affects the local peripheral nervous system and prevents transmission of pain signals to the brain by allowing the GABA receptors to function properly. As a result, the patient obtains pain relief without the unfortunate side effects usually encountered with conventional pain medications. It should be noted that GABA is not a topical anesthetic like lidocaine and the other “-caine” suffix compounds such as benzocaine, xylocaine and the like and does not exhibit its pain-relieving properties in that manner.

The GABA present in the skin compositions works on the peripheral nervous system as well as the skin, fascia and the underlying muscle. By increasing the amount of GABA by application of this formulation the receptors work on local nerve axons to inhibit electrical activity and the level of pain decreases. Additionally, endogenous GABA has demonstrated scientifically proven properties of blocking inflammation separate and independent of its effect as the body's main inhibitory neurotransmitter. Application of exogenous GABA locally, increases the availability of GABA to decrease inflammation where it is applied.

Symptomatic relief is usually obtained more quickly from the systemic, intranasal or oral route than local relief is obtained from local topical application. For example, patients often obtain relief as soon as 15 seconds to 2 or 3 minutes after the initial nasal spray or transbuccal application whereas the topical approach to a local site usually takes effect between 2-20 minutes after application.

### Therapeutically Effective Amounts of GABA

The compositions containing GABA for use in the present invention generally comprise a therapeutically effective amount of GABA when a reasonable volume of the composition is applied using the routes of administration described herein. Actual amounts used may vary according to the severity and type of pain.

A therapeutically effective amount of GABA when used in semi-solids such as ointments, gels, pastes, creams, salves and the like in topical skin applications, suitably include from about 100 to about 300 mg, preferably about 150 to about 250 mg GABA, per 10 square cm of applied surface. Clinically, we use about 200 mg GABA per 10 square cm of application site surface quite successfully from once per day to 4-6 times per day as needed.

Therapeutically effective amounts of GABA for the nasal, sublingual, transpalatal or transbuccal route per administration include from about 10 mg to about 80 mg and preferably from about 20 mg to about 60 mg and most preferably from about 30 mg to about 50 mg per application. Clinically, for intranasal administration, we use about 40 mg per application, divided into two dosages, one for each nostril, quite successfully. Amounts outside the upper and lower ends may be used if tolerated.

The effective dosage regimen can be administered as frequently as needed, depending upon the severity of the pain, from once per day to every 4-6 hours, daily as needed.

### Concentration of GABA

The concentration of GABA in the compositions applied suitably includes:

for liquids, an amount of GABA from about 5% to about 25% or more, GABA by weight and preferably from about 8% to about 12% GABA by weight of solution, (about 50 mg to about 250 mg GABA per ml, and about 80 mg to about 120 mg GABA per ml, respectively). These would be suitable for nasal sprays, buccal tissue sprays, nose drops and sublingual drops, usually 3-4 times per day. It is preferred to avoid amounts at the high end of the range and higher because irritation of the tissue may occur. Amounts at the low end may tend to be less efficacious.

A preferred formulation for the nasal spray and the preferred amounts of GABA in a nasal spray is usually about 100 mg GABA per ml of solution, i.e. about 10% GABA per ml with an application dosage spray of about 40 mg GABA from a spray volume of about 0.4 ml, (4 sprays of 0.1 ml volume per application) usually 3-4 times daily.

For the nasal spray, a delivered dose of about 20 mg of GABA into each nostril, will increase the natural blood level of endogenous GABA significantly. Once the spray dries, the residual GABA will adhere to mucosal membranes continuing to be absorbed thus keeping blood levels elevated for enough time for the mechanisms in the brain to read these elevations. A GABA concentration in the nasal spray of about 100 mg per ml will deliver the recommended 40 mg of GABA with two 0.1 ml metered sprays into each nostril.

For semi-solids and other skin formulation type compositions such as pastes, creams, gels, salves, ointments and the like, the concentration of GABA suitably ranges from about 100 mg to about 300 mg per ml of composition, i.e. from about 10% to about 30% by weight per ml, and preferably from about 150 mg to about 250 mg GABA per ml of composition, i.e., from about 15% to about 25% by weight per ml. The compositions are effectively rubbed into the affected site for about 2 to 3 minutes or so and allowed to air dry.

As can be seen from the foregoing, the methods of the invention address the major disadvantages of pain medication which involves absorption into the central nervous system. With this invention there is rapid relief with virtually no impact on cognitive function or other CNS generated side effects.

Pain relief is virtually immediate and does not require the processing time for oral medications. The invention is useful for acute pain as well as for chronic pain.

Persons with chronic pain who have been treated with the methods of the invention have experienced immediate long term pain relief with little to no adverse side effects. Many of the patients using this medication for a period over months have reported greatly improved quality of life and reduced or eliminated dependence on oral pain medicines.

Typically, patients usually follow a regimen of application every 4-6 hours as needed. No significant adverse effects have been observed upon prolonged use of several months or more.

Over the course of developing the methods of the present invention, we have used topical formulations in the form of creams of various compositions in tests on a large group of patients with persistent pain from various causes. In the vast majority of cases, the relief was immediate and substantial. The reduction in standard subjective pain scales in ninety-two (92) patients showed greater than fifty (50) percent reduction of pain within twenty minutes on 82.61% of the patients and 100% pain relief in 45.65% of the patients. These patients experienced far more rapid and profound pain relief than any other oral, topical or transdermal medication and none reported any significant side effects.

### EXAMPLES

A. The following patients having the starting pain level noted, were administered the topical composition containing GABA described in paragraph [0073]. Pain levels were expressed at 2, 5 and 20 minutes after dosing as shown below with the Results indicated.

| Patient | Start Pain | Two | Five | Twenty | | Percent |
|---|---|---|---|---|---|---|
| (Age) | Score | Minutes | Minutes | Minutes | Change | Change |
| D A (58) | 10 | 2 | 0 | 0 | 10 | 100.00 |
| N M (59) | 6 | 3 | 1 | 0 | 6 | 100.00 |
| T S (62) | 9 | | 0 | 0 | 9 | 100.00 |
| P B (52) | 8 | 2 | 0 | 0 | 8 | 100.00 |
| A M (34) | 8 | 0 | | 0 | 8 | 100.00 |
| F T (63) | 5 | 0 | | 0 | 5 | 100.00 |
| D S (43) | 4.5 | 0 | | 0 | 4.5 | 100.00 |
| J R (82) | 6 | 3 | 0 | 0 | 6 | 100.00 |

### Results

Long Term Response
DA—reduces chronic low back and leg pain level to 0/10 consistently-reduced opioids
NM—reduces chronic neck and low backpain level to 0/10 consistently-reduced opioids
TS—keeps Neuropathic pain and Complex Regional Pain Syndrome Pain manageable and reduces it to 2/10-reduced opioids
PB—lowers fibromyalgia pain, chronic degenerative neck pain and chronic degenerative back injury pain by half consistently-reduced opioids
AM—reduced overall chronic post-infectious neuropathic pain level consistently, helps with flares
FT—Average chronic degenerative back and neuropathic pain level dropped from a 6.5 to 3 in a year, no injections for pain in a year
DS—reduced degenerative thoracic and lumbar and neuropathic pain from an average of 6 to an average of 1.5, more active, reduced opioids
JR—Reduced her chronic back pain from 6 to 0 consistently

The above results dramatically demonstrate the alleviation and elimination of pain using the invention.

The topical cream formulation was prepared from the following:
GABA—20% by weight per ml of final composition.
CREAM BASE—80% by weight per ml of equal weights of LIPODERM® and VAN PEN®, two skin formulations available commercially from PCCA Corp. of Houston, Tex. and containing primarily the ingredients set forth in paragraph [0033] hereof
The GABA is dispersed in ethylene glycol to form a thickened liquid which is homogeneously blended with the CREAM BASE to form the topical cream formulation.

The amount of cream used on each patient was one ml per 10 square cm of applied skin surface. The ratio of LIPODERM® to VAN PEN® may be adjusted to provide varying cream consistencies.

B. The following patients were administered nasal spray application of a 10% or 20% GABA formulation as shown below. The composition of the nasal spray used on the patients was:

GABA—100 mg/ml

Sodium Bisulfite—1 mg/ml

Methyl Paraben—0.5 mg/ml

Propyl Paraben—0.3 mg/ml

Sterile Water (injectable grade)—1 ml per 100 mg of GABA

The patient was instructed to apply 2 sprays per nostril as needed.

The sprayer had a volume of 0.1 ml per spray.

| | | Start Pain | End Pain | | | Percent |
|---|---|---|---|---|---|---|
| Name | Age | Score | Score | Minutes | Change | Change |
| M S | 69-10% | 8 | 0 | 5 | 8 | 100% |
| A L | 65-20% | 8 | 1 | 2 | 7 | 88% |
| B Y | 59-20% | 7 | 2 | 15 | 5 | 71% |
| G W | 66-10% | 8 | 2 | 3 | 6 | 75% |
| J L | 60-10% | 6 | 2 | 4 | 4 | 67% |
| E R | 48-10% | 8 | 2 | 2 | 6 | 75% |
| H W-G | 56-10% | 7 | 0 | 3 | 7 | 100% |
| N M | 36-10% | 7 | 3 | 2 | 4 | 57% |
| I K | 93-10% | 8 | 0 | 2 | 8 | 100% |

Long Term Use
MS—Whenever she uses the nasal spray her pain goes to 0/10 and 5 years of leg numbness and tingling stops. Quality of life is vastly improved.
AL—Helps with acute shoulder pain, but reduces chronic low back pain to 0/10 with long lasting relief, reduces stress and depression.
BY-25 years of chronic 8/10 abdominal pain is usually 0/10 all day long.
   If she has any pain it goes away in minutes with a spray.
   Anxiety is markedly decreased.
GW—Year long hip pain after breaking her hip, disappeared after a week of use, Anxiety is markedly decreased.
JL—Chronic knee and fibromyalgia pain has gone from 6/10 to 0.5 out of 10 after 6 years of severe pain.
ER—Pain is dramatically reduced with excellent mood improvement.
HW-G—Dramatic decrease in pain despite having to move from Hawaii to California and going through a very hard divorce.
NM—Reduces pain of all levels, despite years of arthritic pain and fibromyalgia.
IK—Severe spinal stenosis pain reduced to 0 every time he sprays with very rare exception.

Consistent experience with the long term use of nasal spray is that it has a sustained effect that improves over time. People using it see more profound pain relief and several people who did not initially respond had excellent pain relief within one week to one month of three or four times daily use.

The foregoing tests show the efficacy of the nasal spray formulations and the ability to reduce pain in the treated patients.

What is claimed is:

1. The method for systemically alleviating pain in a subject suffering from pain which comprises administering to said subject a therapeutically effective amount of exogenous GABA by contacting epithelial tissue of the subject with a composition comprising GABA and allowing a therapeutically effective amount of GABA to be absorbed by the epithelial tissue.

2. The method of claim 1, wherein the subject is human.

3. The method of claim 2, wherein the epithelial tissue is selected from the group consisting of skin, nasal mucosa, oral mucosa, buccal, palatal, sublingual, rectal, vaginal, intrathecal, intramuscular and intravenous.

4. The method of claim 3, whereas the epithelial tissue is nasal mucosa.

5. The method of claim 4, wherein the GABA is present in a nasal spray composition.

6. The method of claim 5, wherein the amount of GABA applied per treatment ranges from about 10 mg to about 80 mg of GABA.

7. The method of claim 6, wherein the amount of GABA present in said nasal spray is in the range from about 50 mg to about 250 mg per ml of nasal spray solution.

8. The method of claim 7, wherein the composition is administered daily and a daily dose is from about 30 mg to about 320 mg of GABA.

9. The method of claim 5, wherein the GABA is applied to a patient in a single or multiple daily dosage regimen.

10. The method of claim 6, wherein the GABA dose per treatment is from about 30 mg to about 50 mg of GABA.

11. The method of claim 9, wherein the composition is administered 3-4 times daily.

12. The method of claim 1, wherein the epithelial tissue is skin.

13. The method of claim 12, wherein the subject is human.

14. The method of claim 13, wherein the GABA is present in a topical skin composition.

15. The method of claim 14, wherein the topical skin composition is selected from the group consisting of creams, gels, ointments, pastes, salves and lotions.

16. The method of claim 14, wherein the amount of GABA applied per treatment ranges from about 100 mg to about 300 mg of GABA per 10 square cm of application site.

17. The method of claim 16, wherein the topical composition comprises from about 10% to about 30% by GABA based on the weight of total composition.

18. The method of claim 14, wherein the composition is administered daily and a daily dose is from about 200 mg to about 1200 mg of the GABA per 10 square cm of application site.

19. The method of claim 18, wherein the daily dose is about 150 mg to about 300 mg of the GABA ingredient per application.

20. The method of claim 1 wherein the composition comprising of GABA has a consistency ranging from water to free flowing liquids of higher viscosity to semi-solids to solids which can be applied to the skin.
